(19) United States
(12) Patent Application Publication
(10) Pub. No.: US 2022/0146459 A1
(43) Pub. Date: May 12, 2022

(54) GAS DETECTION SYSTEM

(71) Applicants: Will JOHNSON, Saline, MI (US); Michigan Aerospace Corporation, Ypsilanti, MI (US)

(72) Inventor: Will JOHNSON, Saline, MI (US)

(21) Appl. No.: 17/436,572

(22) PCT Filed: Mar. 6, 2020
(86) PCT No.: PCT/US2020/021441

§ 371 Date: Sep. 3, 2021

Related U.S. Application Data
(60) Provisional application No. 62/814,471, filed on Mar. 6, 2019.

(51) Int. Cl. G01N 29/24; G01N 33/00

(52) U.S. Cl. CPC G01N 29/24; G01N 2291/02809; G01N 2291/021; G01N 33/0027

(57) ABSTRACT

A system and method for detecting gas sensing employs a waveguide for both electromagnetic and acoustic radiation. Electromagnetic radiation is intensity-modulated and introduced into a waveguide. The waveguide is perforated to admit a gas that absorbs energy at the wavelength of the electromagnetic radiation. An acoustic wave is produced in the waveguide with a frequency equal to the modulation frequency of the electromagnetic radiation. The acoustic wave is received by an acoustic sensor and the presence of the gas is determined in accordance with the received acoustic wave.

### CROSS REFERENCE TO RELATED APPLICATIONS

This application is a national stage application filed under 35 U.S.C. § 371, of International Patent Application No. PCT/US2020/021441, filed on Mar. 6, 2020, which claims priority to U.S. Provisional Application No. 62/814,471, filed on Mar. 6, 2019, both of which are hereby incorporated by reference in their entireties.

This application claims priority to U.S. Provisional Patent Application No. 62/814,471, filed on Mar. 6, 2019, the entire contents of which are hereby incorporated herein by reference.

A system is described in the following for detecting the presence and/or measuring the concentration of a gas with a combination of electromagnetic and acoustical sensing.

### BACKGROUND OF THE INVENTION

Monitoring for the presence or concentration of a gas such as methane is a common need for many industrial applications. For example, there may be a need to measure the presence or concentration of a gas that may be leaking from a single or multiple locations in a very large area such as a carbon sequestration site or along a gas pipeline. In these scenarios, a distributed grid of in situ sensors may be both expensive and logistically challenging to deliver power and obtain data from all sensors. Some remote sensing methods using, e.g., differential absorption LIDAR, can scan over large areas. However, these systems may be limited in range, often to a few kilometers, and are quite expensive typically.

In accordance with the photoacoustic effect, sound waves may be formed due to light absorption in a sample. In some systems, the light may be modulated in intensity at an acoustic frequency (e.g., 10 kHz). If passed through a trace gas, the trace gas may absorb a fraction of the optical energy and the gas may increase in temperature from this energy. This increase in temperature causes the gas to expand, and when the light is turned off, the gas cools and contracts. By modulating the light source, the trace gas can be made to expand and contract such that the modulation frequency produces sound waves that can be detected with an acoustic sensor such as a microphone. When the trace gas concentration is large, more light is absorbed and the sound is loud (i.e., the magnitude of the acoustic signal is large); when the concentration is small, less energy is absorbed and the sound produced is quieter (the magnitude is smaller).

The range of volumes can be calibrated to the range of gas concentrations, allowing a user to directly measure a gas concentration from the magnitude of the acoustic response. This method is limited to short ranges, however, since the sound produced radiates in all directions, limiting the amount of acoustical energy that can be collected and sensed.

### SUMMARY OF THE INVENTION

As more fully described below, a photoacoustic response in a gas can be generated with electromagnetic radiation in a waveguide that functions as both an electromagnetic radiation waveguide and an acoustic waveguide. In one or more embodiments, the electromagnetic radiation is microwave radiation and the waveguide carries the acoustic wave to an acoustic detector.

In accordance with at least one embodiment, a gas sensing system comprises an emitter of electromagnetic radiation; a waveguide; and an acoustic receiver; wherein the emitter is controlled to emit the electromagnetic radiation to the waveguide with a wavelength selected to be absorbed by a gas in the waveguide; the emitter is configured to modulate the electromagnetic radiation at the emitted wavelength with a modulation frequency and produce an acoustic wave in the gas in the waveguide with an acoustical frequency equal to the modulation frequency; and the acoustic receiver is arranged to receive the acoustic wave via the waveguide.

In accordance with at least one other embodiment, a method for detecting a gas comprises introducing electromagnetic radiation to a waveguide, the electromagnetic radiation having a wavelength that is absorbed by a gas in the waveguide; modulating the electromagnetic radiation with a modulation frequency; producing an acoustic wave in the gas in the waveguide with an acoustical frequency equal to the modulation frequency; receiving the acoustic wave; and detecting the gas in accordance with the received acoustic wave.

### BRIEF DESCRIPTION OF THE DRAWINGS

FIG. 1 illustrates features of a gas detection system in accordance with one or more embodiments described herein.

FIG. 2 illustrates an example of a gas detection system in which a waveguide is collocated with a gas pipeline to detect a gas leak.

FIG. 3 illustrates an example of a gas detection system in which a waveguide is located within or proximate to a refinery to act as multiple gas sensors at once.

### DETAILED DESCRIPTION OF THE INVENTION

The following description presents one or more embodiments of a gas detection system useful for monitoring and/or measuring for the presence and concentration of trace gases. Without limitation, examples are given of a distributed gas detection system, but the person of ordinary skill in the art will readily recognize that the principles and features described herein may be equally applicable to the detection of gas in a more localized system. Furthermore, one or more embodiments are described in which the electromagnetic radiation that is utilized to produce acoustic waves is microwave radiation. However, in accordance with the principles of the invention, other wavelengths may be used as suitable for the intended purpose.

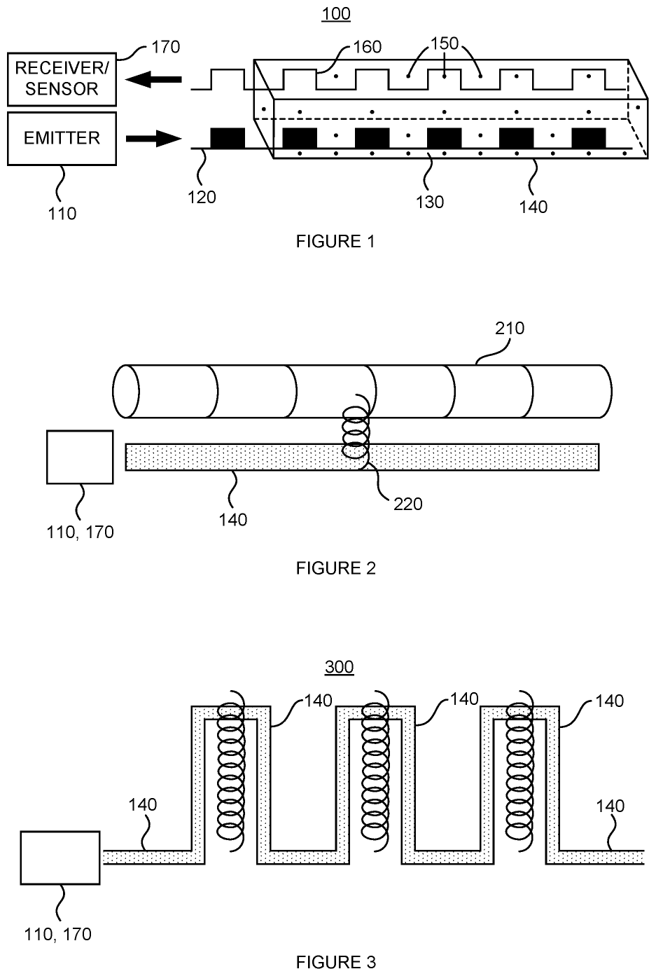

FIG. 1 illustrates features of a gas detection system 100 in accordance with one or more embodiments described herein. As shown in FIG. 1, a microwave emitter 110 is configured and positioned to emit microwave radiation (microwaves) 120 of a desired wavelength and intensity. In one or more embodiments, the wavelength of the microwave radiation 120 is selected based on which wavelength is strongly absorbed by the gas of interest, such as gas 130.

Microwave radiation 120 is introduced into a waveguide 140 from the emitter 110, which is provided with perforations 150 to allow gas 130 to leak into the waveguide via perforations 150. To this end, perforations 150 should be sufficiently large to permit entry of gas 130 but small enough to not affect the wave guiding properties of waveguide 140. Based on the selected wavelength, waveguide 130 may be constructed of appropriate cross-sectional dimensions and material having properties depending on the waveguide chosen, such as dielectric properties, to function as an efficient waveguide for microwave radiation 120. Some example wavelengths and their associated dimensions include for a common 2.4 GHz RF carrier frequency a rectangular waveguide would measure 3.8×1.9 inches, or for a 60 GHz RF carrier frequency a rectangular waveguide would measure 0.15×0.075 inches in cross section.

Depending on the microwave properties, the gas 130 may absorb a fraction of the energy of the microwave radiation 120, causing the gas 130 to increase in temperature in proportion to the absorbed energy. This increase in temperature causes the gas 130 to expand, and when emission is stopped, the gas cools and contracts. By modulating the intensity of microwave radiation 120 emission, the gas 130 can be made to expand and contract at a desired modulation frequency and produce one or more acoustic waves or signals 160 of a frequency equal to the modulation frequency. Such an acoustic signal can be detected with, e.g., an acoustic sensor such as a microphone. When the concentration of gas 130 is large, more radiation energy is absorbed, resulting in a correspondingly higher intensity of the acoustic wave 160 (e.g., a “louder sound”). Conversely, when the concentration is smaller, less energy is absorbed and the acoustic wave 160 produced is correspondingly smaller in intensity (e.g., a “quieter sound”). The range of acoustic intensities (e.g., sound volumes) can be tailored to the concentration range of gas 130, allowing the concentration of gas 130 to be determined from the magnitude of the acoustic response (i.e., 10 dB volume for 100 parts per million of gas concentration).

Microwave radiation 120 may be modulated in intensity at an acoustical frequency at which waveguide 140 can also efficiently function as an acoustical waveguide if constructed with an appropriate cross-sectional geometry. Such a waveguide may efficiently guide at least some of the acoustical energy along its length to an acoustic detector 170 located at one or both ends of the waveguide (FIG. 1 shows an example in which acoustic detector 170 is located at the microwave radiation emitting end of waveguide 140). By guiding the acoustic wave 160, the acoustic energy can be carried over great distances and still be detectable. In addition, although microwave radiation 120 may be emitted continuously, if the intensity-modulated microwave signal 120 is emitted in a burst, the time delay between the burst and the reception of the acoustic signal may provide information indicating how far along waveguide 140 the gas 130 is present, as well as the magnitude of the acoustic signal. From this data, information about the gas concentration can also be determined.

As an example, as shown in FIG. 2, the waveguide 140 can be collocated with a gas pipeline 210 to detect a leak 220 of the gas 130. Thus, with even a single electromagnetic source and a single acoustic detector coupled to an appropriately dimensioned waveguide, the location and concentration of a gas along a variety of lengths, even great lengths (i.e., hundreds to thousands of meters), can be determined.

As another example, as shown in FIG. 3, the waveguide 140 can be located within or proximate to a refinery 300 to act as multiple gas sensors at once. An advantage of the disclosed system using microwave wavelengths is that the surface finish quality on waveguide 140 need not be so high as might be required for other wavelengths, allowing basic tubing or ductwork of appropriate size to act as waveguide 140. Low-cost waveguide material combined with even a single emitter and a single receiver (or single emitter/receiver) can reduce the cost of measuring large areas and the system can be readily built to measure a variety of gases of interest provided the gas has a sufficiently strong absorption feature at available wavelengths. Additionally, the emitter and receiver arrangement may simplify the logistics of measuring over long lengths or at multiple points of possible gas presence since power need only be provided from one location and all data can be read from a single location in one or more embodiments.

In analyzing the sensed data, if the system is configured to not trying to resolve absolute concentration and position along the waveguide, the presence or absence of the gas of interest is determined by just listening out for a tone. For an embodiment configured to determine concentration and distance along the waveguide when the gas is present, the time of arrival of the acoustic waves relative to the electromagnetic waves is used to calculate the distance along the waveguide the gas is present (knowing the speed of sound) and the concentration will be calculated using calibration data generated ahead of time for the sensor that maps the acoustic volume at a waveguide distance to a gas concentration

In another variation of the invention, more than one type of gas could be detected at the same time using a plurality of sources whose individual wavelengths are absorbable by the gases to be detected. In addition, the waveguide would have to be dimensioned to be able to guide two or more wavelengths simultaneously.

Regarding the design of the waveguide itself, in one embodiment, the waveguide may be constructed flexible metal tubing similar to flexible dryer vent ductwork, or a metal screen mesh lining a tubing of arbitrary material that can be shaped to direct the waveguide through the area of interest. The metal mesh will act as the reflector for the electromagnetic energy while allowing gas to perforate through. The length may be determined based on what is required by the application in combination with how long a single emitter/receiver can measure (i.e., an application may require 10 km of sensing, but a single system may only measure 5 km requiring two systems to cover the full range).

Generally, in operation, the waveguide is positioned as close as is practical to the emission source to be monitored. Ideally, the waveguide should be connected to the pipeline, but if attaching directly is not practical, it must be close enough to be exposed to reasonable concentrations before the environment dilutes and carries the gas away (i.e., via the wind) where the sensor cannot interact with the gas.

The length of the waveguide is driven by the application, but would likely be selected to be as long as possible for pipeline monitoring where entire states need monitoring. With more localized monitoring, such as the inside of a refinery, the length will be as much as is needed to sense all locations of interest.

Regarding the selection of a waveguide to use, while the fundamental dimensions get driven by the frequencies chosen to detect the gas, the cross-sectional dimension of the waveguide will be driven by the wavelength selected to absorb a specific gas. In addition, the exact material selection (i.e., vinyl tubing with a stainless steel mesh vs an aluminum flexible tube) will be driven by numerous factors including but not limited to cost and practicality.

With respect to the design of the waveguide for purposes of perforation, the waveguide may be designed to use a diffusive material such as a metal screen or a dielectric material with good waveguiding properties that has a plurality of holes physically defined on its surface. The size of the perforations will be defined to be small enough to not allow leakage of electromagnetic radiation (which will be calculated based on the wavelength chosen to detect the gas of interest), but large enough to allow the gas of interest to diffuse through it. The number of perforations must be high enough to make diffusion efficient and covering the entire length of the waveguide to make all parts of the waveguide sensitive to gas infiltration.

Insofar as the selection of the wavelength of the electromagnetic energy, other wavelengths may be used. All that is required is that the gas of interest be able to absorb the wavelength being used and then the waveguide has its dimensions matched to the wavelength used. In determining the amount or frequency of modulation, the size of the waveguide determines the acoustic frequency. As such, the designing of the waveguide takes into consideration the identification of a frequency that the gas of interest absorbs. The geometry required to efficiently guide the electromagnetic energy is then calculated, and then a modulation frequency is selected wherein the now chosen waveguide dimensions will guide acoustically with a high level of efficiency.

It should be noted that the present invention is designed to operate with particular gas or gasses. The invention can be used to quantify gas concentration and location of a certain gas of interest. Provided the wavelength is selected that only the gas of interest absorbs, a response from the invention will be unambiguous as to whether the gas of interest is present or another gas is in the tube (the other gas won't produce a response since it doesn't absorb at the wavelength chosen).

With respect to the design and construction of the emitter and receiver (detector), for longer range measurements (i.e., hundreds to thousands of meters), the electromagnetic and acoustic radiation cannot be amplified inline in the waveguide once generated. For longer distance measurements where a single emitter is insufficient, multiple emitters distributed along the waveguide would be used.

Embodiments of a distributed gas detection system have been described in the context of a gas detector that uses modulated microwave radiation and an appropriately constructed waveguide to generate acoustic waves from which to detect the presence and/or concentration of a gas. The described embodiments do not require a complex and expensive distributed grid of in situ sensors to deliver power to and obtain data from all sensors routinely. These and other advantages are merely illustrative and the disclosed embodiments may enjoy one or more of these advantages as well as other advantages. Moreover, the disclosed gas distribution detection system is not limited to detecting any particular type of gas, but may be used to detect a variety of gases. All that is required is the gas of interest be able to absorb the wavelength being used and the waveguide be designed to have its dimensions matched to the wavelength used.

Various changes and modifications to the disclosed gas distribution detection system will be apparent to those skilled in the art. All such changes and modifications that rely on the basic teachings and principles through which the invention has advanced the state of the art are to be understood as included within the spirit scope of the present invention.

What is claimed is:

1. A gas sensing system, comprising:
an emitter of electromagnetic radiation;
a waveguide; and
an acoustic receiver;
wherein the emitter is controlled to emit the electromagnetic radiation to the waveguide with a wavelength selected to be absorbed by a gas in the waveguide;
wherein the emitter is configured to modulate the electromagnetic radiation at the emitted wavelength with a modulation frequency and produce an acoustic wave in the gas in the waveguide with an acoustical frequency equal to the modulation frequency; and
wherein the acoustic receiver is arranged to receive the acoustic wave via the waveguide.

2. The system of claim 1, wherein the waveguide has perforations to introduce the gas into the waveguide.

3. The system of claim 1, wherein the electromagnetic wave radiation is microwave radiation.

4. A method for detecting a gas comprising:
introducing electromagnetic radiation to a waveguide, the electromagnetic radiation having a wavelength that is absorbed by a gas in the waveguide;
modulating the electromagnetic radiation with a modulation frequency;
producing an acoustic wave in the gas in the waveguide with an acoustical frequency equal to the modulation frequency;
receiving the acoustic wave; and
detecting the gas in accordance with the received acoustic wave.

5. The method of claim 4, wherein the electromagnetic radiation is emitted to the waveguide from a single emitter; and
wherein the acoustic wave is detected with a single acoustic receiver.

6. The method of claim 4, further comprising:
emitting the electromagnetic wave radiation in a burst;
determining the time between emission of the burst and receiving the acoustic wave; and
determining a concentration of the gas in accordance with the determined time.
